(19) United States
(12) Patent Application Publication
(10) Pub. No.: US 2005/0159722 A1
(43) Pub. Date: Jul. 21, 2005

(54) Wearing article provided with bamboo carbon material

(75) Inventor: Sen-Mei Cheng, Changhua Hsien (TW)

Correspondence Address: TROXELL LAW OFFICE PLLC, SUITE 1404, 5205 LEESBURG PIKE, FALLS CHURCH, VA, 22041, US

(73) Assignee: Taiwan Paiho Limited

(21) Appl. No.: 10/757,602

(22) Filed: Jan. 15, 2004

(51) Int. Cl. A61F 13/15; A61F 13/20

(52) U.S. Cl. 604/367

(57) ABSTRACT

A wearing article provided with bamboo carbon material includes a disposable wearing article like a diaper for babies which is mainly made up of a belly pad section, a thin film with diagrams defined thereon attached at the front side of the belly pad section thereto, and a wrapping section with two buckling pieces attached at both lateral sides thereof to be fastened to the thin film respectively thereby. The diagrams of the thin film are provided with dark ink into which, along with a fastening agent layer laminated at a releasing paper and the buckling piece there-between, bamboo carbon powder is added. The bamboo carbon powder is made of carbonized old bamboo grown for over 4 years and burned at a high temperature before ground into powder for use. Via the bamboo carbon powder of strong adhesive and dissolving capacities, the disposable wearing article thereof is equipped with the functions of anti-bacteria, humid-adjustment, and deodorization to easily absorb and dissolve some harmful chemicals, or defeat bugs. Besides, infrared suitable to the absorption of human body for good blood circulation and body health is produced, and beneficial negative ions are increased in the air to balance the humidity and achieve anti-bug design, efficiently boosting the quality and effects of the disposable wearing article thereof.

### BACKGROUND OF THE INVENTION

The present invention is related to a wearing article provided with bamboo carbon material, comprising a disposable wearing article like a diaper for babies having a thin film with diagrams defined thereon attached at the front side of a belly pad section thereto, and two buckling pieces attached at both lateral sides of a wrapping section thereof to be fastened to the thin film respectively thereby. The diagrams of the thin film is provided with dark ink into which, along with a fastening agent layer laminated at a releasing paper and the buckling piece there-between, is added bamboo carbon powder of strong adhesive and dissolving capacities; whereby, the disposable wearing article is equipped with the functions of anti-bacteria, humid-adjustment, and deodorization to easily absorb and dissolve some harmful chemicals or defeat bugs. Meanwhile, infrared suitable to the absorption of human body for good blood circulation and body health is produced, and beneficial negative ions are increased in the air to balance the humidity and achieve anti-bugs design, efficiently boosting the quality and effects of the disposable wearing article thereof.

A conventional disposable wearing article such as a diaper is provided with the functions of anti-bacteria and good air penetrability. But, in spite of that, such conventional disposable wearing article thereof is not equipped with strong adhesive and dissolving capacities, which makes it unable to adjust the humidity and defeat harmful chemicals and bugs in the environment, or to produce beneficial infrared suitable to the absorption of human body for good blood circulation or body health.

### SUMMARY OF THE PRESENT INVENTION

It is, therefore, the primary purpose of the present invention to provide a wearing article provided with bamboo carbon material, comprising a disposable wearing article like a diaper for babies having a thin film with diagrams defined thereon attached at the front side of a belly pad section thereto, and two buckling pieces attached at both lateral sides of a wrapping section thereof to be fastened to the thin film respectively thereby wherein the diagrams of the thin film is provided with dark ink into which, along with a fastening agent layer laminated at a releasing paper and the buckling piece there-between, is added bamboo carbon powder of strong adhesive and dissolving capacities; whereby, the disposable wearing article is equipped with the functions of anti-bacteria, humid-adjustment, and deodorization to easily absorb and dissolve some harmful chemicals or defeat bugs. Meanwhile, infrared suitable to the absorption of human body for good blood circulation and body health is produced, and beneficial negative ions are increased in the air to balance the humidity and achieve anti-bugs design, efficiently boosting the quality and effects of the disposable wearing article thereof.

### BRIEF DESCRIPTION OF THE DRAWINGS

FIG. 1 is a perspective exploded view of the present invention.

FIG. 2 is an enlarged cross sectional view of a thin film of the present invention.

FIG. 3 is an enlarged cross sectional view of a buckling piece of the present invention.

### DETAILED DESCRIPTION OF THE PREFERRED EMBODIMENTS

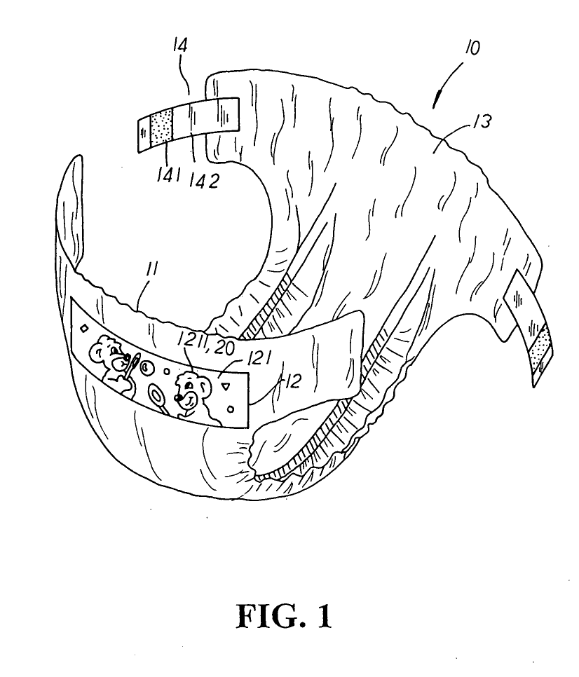

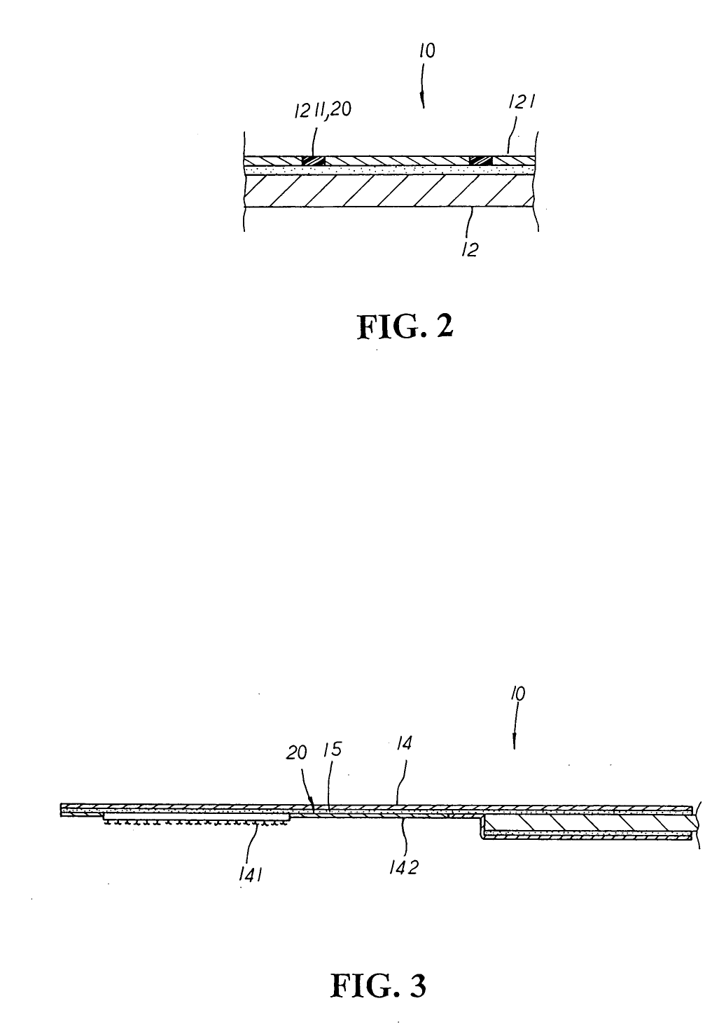

Please refer to FIG. 1. The present invention is related to a wearing article provided with bamboo carbon material, comprising a disposable wearing article 10 like a diaper for babies which is mainly made up of a belly pad section 11, a thin film 12 with diagrams 121 defined thereon attached at the front side of the belly pad section 11 thereto, and a wrapping section 13 with two buckling pieces 14 joined at both lateral sides thereof to be fastened to the thin film 12 respectively in buckling engagement thereby. The diagrams 121 of the thin film 12 are provided with dark ink 1211 having bamboo carbon powder 20 added therein as shown in FIG. 2. A releasing paper 142 with a fastening facet 141 attached at one side thereon is applied to the inner lateral side of the buckling piece 14 thereof, and a fastening agent layer 15 with bamboo carbon powder 20 added therein is laminated at the corresponding inner side of the buckling piece 14 and the releasing paper 142 there-between as shown in FIG. 3. The bamboo carbon powder 20 is made of carbonized old bamboo grown for over 4 years and burned at a high temperature before ground into powder for use. Via the bamboo carbon powder 20 with strong adhesive and dissolving capacities, the disposable wearing article 10 is equipped with the functions of anti-bacteria, humid-adjustment, and deodorization to easily absorb and dissolve some harmful chemicals such as sulfide, nitride, methanol, benzene, or carbolic acid, etc. Meanwhile, infrared suitable to the absorption of human body for good blood circulation and body health is produced, and beneficial negative ions are increased in the air to balance the humidity and achieve anti-bug design, efficiently boosting the quality and effects of the disposable wearing article 10 thereof.

1. A wearing article provided with bamboo carbon material, comprising a disposable wearing article like a diaper for babies which is mainly made up of a belly pad section, a thin film with diagrams defined thereon attached at the front side of the belly pad section thereto, and a wrapping section with two buckling pieces attached at both lateral sides thereof to be fastened to the thin film respectively in buckling engagement thereby; the present invention being characterized by that,
the diagrams of the thin film being provided with dark ink having bamboo carbon powder added therein;
a releasing paper with a fastening facet attached at one side thereon being applied to the inner lateral side of the buckling piece thereof, and a fastening agent layer with bamboo carbon powder added therein being laminated at the corresponding inner side of the buckling piece and the releasing paper there-between;
the bamboo carbon powder being made of carbonized old bamboo grown for over 4 years and burned at a high temperature before ground into powder for use;
via the bamboo carbon powder of strong adhesive and dissolving capacities, the disposable wearing article thereof is equipped with the functions of anti-bacteria, humid-adjustment, and deodorization to easily absorb and dissolve some harmful chemicals such as sulfide, nitride, methanol, benzene, or carbolic acid, etc., or defeat bugs; meanwhile, infrared suitable to the absorption of human body for good blood circulation and body health is produced, and beneficial negative ions are increased in the air to balance the humidity and achieve anti-bug design, efficiently boosting the quality and effects of the disposable wearing article thereof.
